(19) United States
(12) Patent Application Publication
(10) Pub. No.: US 2015/0165112 A1
(43) Pub. Date: Jun. 18, 2015

(54) FLU SHOT KIT

(71) Applicant: ASCLEMED USA, INC. dba EnovaChem Manufacturing, Torrance, CA (US)

(72) Inventor: Robert Prentiss Nickell, Torrance, CA (US)

(73) Assignee: ASCLEMED USA, INC. dba EnovaChem Manufacturing, Torrance, CA (US)

(21) Appl. No.: 14/566,461

(22) Filed: Dec. 10, 2014

Related U.S. Application Data
(60) Provisional application No. 61/914,958, filed on Dec. 12, 2013.

(51) Int. Cl. A61M 5/00; C12N 7/00; A61J 1/05; A61K 39/145

(52) U.S. Cl. CPC A61M 5/002; A61K 39/145; C12N 7/00; A61J 1/05; C12N 2760/16034

(57) ABSTRACT

Provided is a kit comprising a vaccine to the influenza virus. The vaccine can be an immunogenic composition that comprises an inactivated influenza virus. The immunogenic compositions elicits can include subtypes A and/or type B of the influenza virus. A container, such as a single dose pre-filled syringe of the vaccine is placed inside of the kit. Another component of the kit can include a CSR (Central Supply Room) wrap. Another component of the kit can include sterile gloves. Another component of the kit can include a bandage. Another component of the kit can include a disinfectant for topical application, such as an alcohol pad. Another component of the kit can include an instruction sheet.

### CROSS-REFERENCE

The present application claims the benefit of U.S. provisional Appl. No. 61/914,958, filed on Dec. 12, 2013, which is INCORPORATED HEREIN BY REFERENCE IN ITS ENTIRETY.

### BACKGROUND Section of the Invention

With the changing reimbursements, types of health care insurance and new regulations with regards to treatment, a medical practitioner and his/her team when treating a patient is under immense time pressures to perform quickly, efficiently and with safety for the patient. If a patient requires a singular or even multiple injections, the medical practitioner must rely on his/her team to first locate the necessary and correct injectable medications, insure they are the correct strength and percentage, insure dating is current, insure the top has not been contaminated, insure the liquid is not crystallized, or containing growth, insure there is sufficient medication in the vial, and insure that the injection has been stored at the proper temperature and humidity as well as all other possible related materials and safety precautions needed to treat the patient. The medical practitioner can lose valuable time trying to not only locate the necessary drugs and materials to treat the patient but as well to insure the compliance, the safety of the patient by preventing contamination. In some instances, not all the drugs and materials may be readily available, in the current exam room, resulting in delayed treatment and possible and potential medical errors. In addition, the medical practitioner or his/her team may make a mistake by incorrectly choosing the drug, by incorrectly identifying the product, not checking the expiration date, not returning it to the proper environment for storage, or not having enough product and thus requiring an additional sterile vial to be opened to inject directly into the veins of a patient.

There is a need in the art for a convenience kit that solves the above mentioned problems in the medical practitioner office.

### SUMMARY SECTION OF THE INVENTION

Provided is a kit comprising a first container and any two or more of the following components placed therein: a) a second container of an immunogenic composition of an influenza virus; b) a disinfectant for topical administration; c) a bandage; d) a pair of gloves; and e) a CSR (Central Supply Room) wrap. The immunogenic composition can comprise an inactivated influenza virus. The immunogenic composition can be a vaccine, The immunogenic composition can contain one or more of subtypes A and type B. The subtypes of influenza A viruses can be (H1N1) and (H3N2). The second container can be a pre-filled syringe. The disinfectant can be an alcohol pad. The alcohol pad can be an isopropyl alcohol pad with 70% concentration. The first container can he a box with a lid. The bandage can be a spot adhesive bandage. The kit can further comprise an instruction sheet.

Provided is a kit comprising a box. with a lid and following components placed therein: a) a pre-filled syringe of an influenza virus vaccine that elicits a protective immune response to subtypes A and type B of the influenza virus; b) an isopropyl alcohol pad; c) a spot adhesive bandage; d) a pair of gloves; and e) a CSR (Central Supply Room) wrap.

### BRIEF DESCRIPTION OF THE FIGURES

FIG. 1 illustrates a prefilled syringe.

FIG. 2 illustrates a box where a plurality of components are placed therein.

FIG. 3 illustrates a glove.

FIG. 4 illustrates an alcohol pad.

FIG. 5 illustrates a package and a spot bandage.

FIG. 6 illustrates a package and a strip bandage.

FIG. 7 illustrates a face mask.

FIG. 8 illustrates an instruction sheet.

FIG. 9 illustrates a CSR wrap.

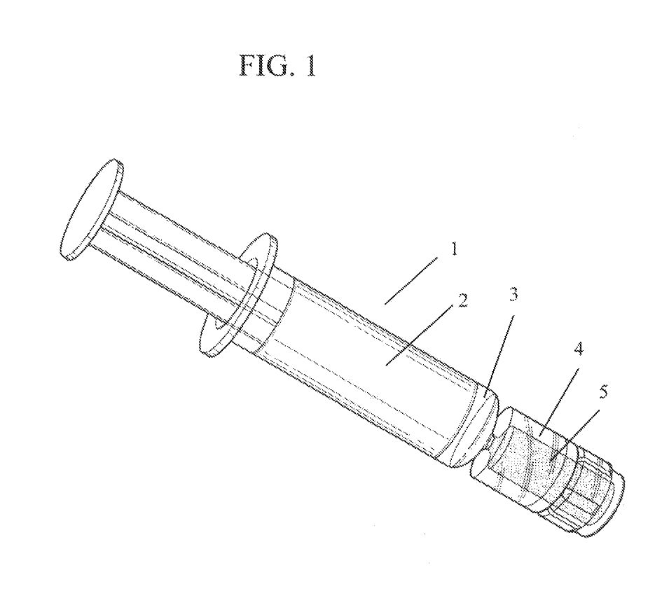

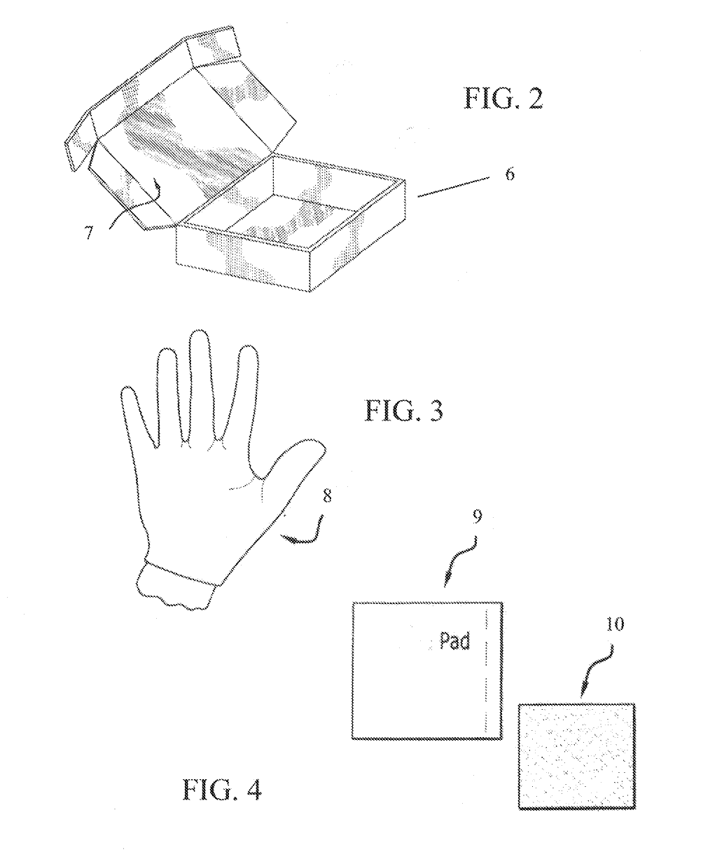

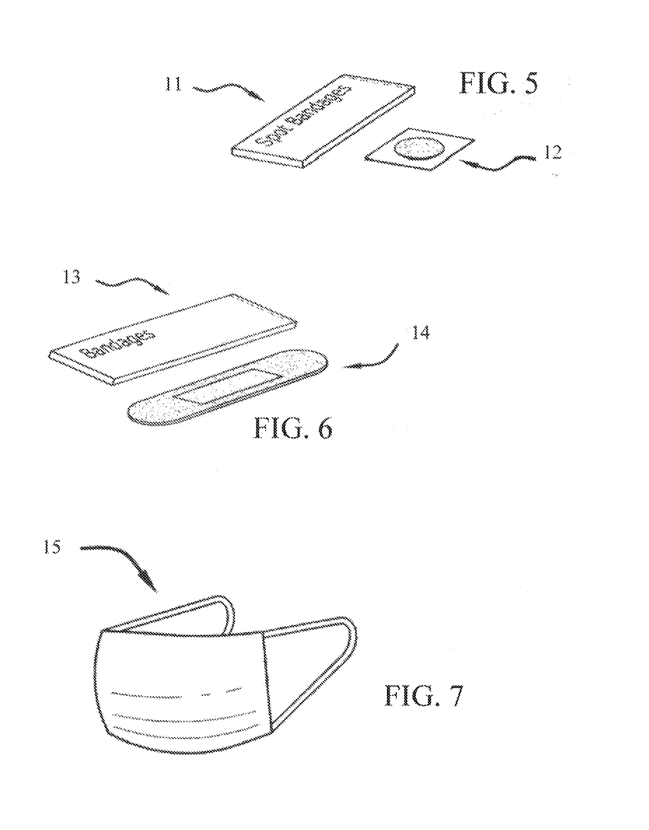

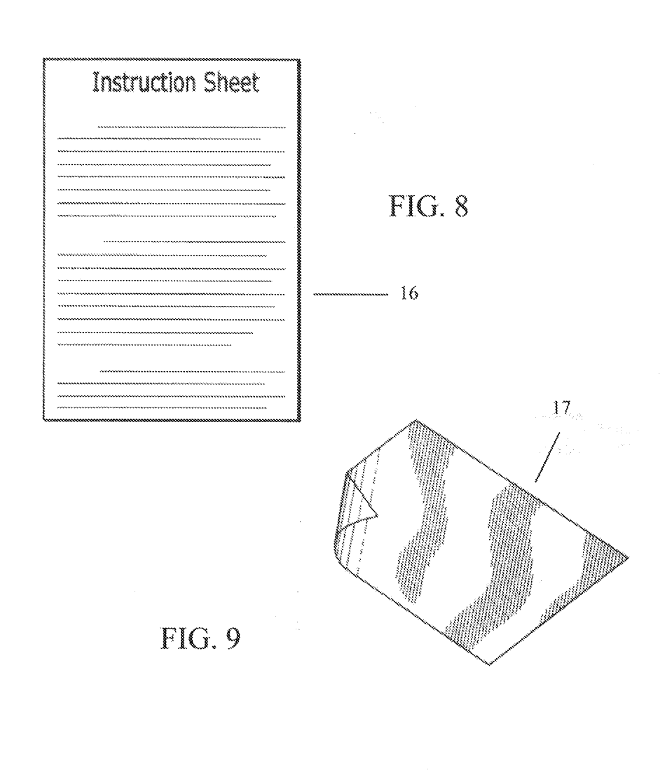

### DETAILED DESCRIPTION OF THE INVENTION

The present invention provides a kit for injection of an influenza virus vaccine.

One component of the kit is an influenza virus vaccine. The kit contains immunogenic composition that comprise one or more antigens of the influenza virus. The influenza virus vaccine, typically in the form of an inactivated influenza virus, can elicit an immunogenic response to subtypes A and/or type B of the influenza virus. Current subtypes of influenza A viruses found in people are influenza A (H1N1) and influenza A (H3N2) viruses. The influenza virus vaccine used in the kit can be FLUVIRIN® or FLUCELVAX®, which contain an inactivated influenza virus vaccine, They are administered as a single 0.5 mL intramuscular injection preferably in the region of the deltoid muscle of the upper arm.

A single-dose 0.5 mL suspension in a pre-filled syringe 1 can be placed in the kit. The pre-filled syringe 1 can come in its own wrapping or box. Cavity 3 of the syringe can be pre-filled with the vaccine. The syringe can have a label 2 with instructions and/or name of the product. The syringe can have a cap 4 that can be removed before use. A needle (not supplied in the kit) can be inserted into receiver 5 of the syringe to create a fluid communication with cavity 3. A medical practitioner would. remove cap 4, insert a needle into the receiver 5, and inject the vaccine.

One component of the kit can be a CSR (Central Supply Room) wrap 17. The CSR wrap 17 can be put on a tray or a surface before placement of the other components of the kit. The CSR wrap 17 keeps a clean environment. The CSR wrap 17 can be in its own wrapping or box.

One component of the medical kit can be a pair of sterile gloves 8. The gloves 8 can be any suitable glove that is used by a medical practitioner, such as latex or nitrile gloves. In one embodiment, a pair of nitrile gloves 8 are part of the kit. Nitrile gloves are a type of disposable glove 8 made of Nitrile rubber, also known as Buna-N, Perbunan, or NBR. Nitrile gloves contain no latex. A medical practitioner can tear a package containing the gloves 8 and wear the gloves 8 before performing an injection to help prevent contamination and increase compliance. The gloves 8 can be in its own wrapping or box.

One component of the medical kit can be a bandage(s) 12, 14. The bandage can be a spot bandage 12, or a bandage in the shape of a strip 14 like a Band-Aid®, or other suitable bandage. The bandage 12, 14 can be in a package 11, 13. A medical practitioner can tear the package 11, 13 and use the bandage 12, 14 on a patient after an injection is performed. Before a bandage 12, 14 is applied, the injection site can be disinfected.

One component of the medical kit can he a disinfectant for topical application, such as an alcohol pad 10, which is an antiseptic for preparation of the skin prior to injection, The alcohol pads 10 can be individually foil wrapped 9, which the medical practitioner would tear to remove the pad 10. The alcohol swabs can contain 70% isopropyl alcohol.

The components of the kit can be placed in a. container, which is typically a box 1. The box 1 can be a rectangular cube which has a lid 2 on top for opening the box.

The kit can contain an instruction sheet 16. The instruction sheet 16 can identify one or more components of the kit, provide instructions as to how to use each component, and optionally suggest an order of use for the components of the kit. The instruction sheet 16 can provide instructions as to where and how to inject each of the active ingredients.

Each component of the kit can he described on the box 6 of the kit, for example on the sides of the box 6. Each component can have its own separate instruction sheet 16 provided by its own manufacturer.

One component can be a mask 15 to help prevent the spread of disease between the patient and the practitioner. The face mask 15 can be placed inside the kit without wrapping.

What is claimed is:

1. A kit comprising a first container and following components placed therein:
a) a second container of an immunogenic composition of an influenza virus;
b) a disinfectant for topical administration;
c) a bandage;
d) a pair of gloves; and
e) a CSR (Central Supply Room) wrap.

2. The kit of claim 1, wherein the immunogenic composition comprises an inactivated influenza virus.

3. The kit of claim 1, wherein the immunogenic composition is a vaccine.

4. The kit of claim 1, wherein the immunogenic composition contains one or more of subtypes A and type B of the influenza virus.

5. The kit of claim 4, wherein the immunogenic composition contains both subtypes A and type B.

6. The kit of claim 5, wherein the subtypes are H1N1 and H3N2.

7. The kit of claim 1, wherein the second container is a pre-filled syringe.

8. The kit of claim 1, wherein the disinfectant is an alcohol pad.

9. The kit of claim 8, wherein the alcohol pad is an isopropyl alcohol pad with 70% concentration.

10. The kit of claim 1, wherein the first container is a box with a lid.

11. The kit of claim 1, wherein the bandage is a spot bandage.

12. The kit of claim 1, further comprising an instruction sheet placed inside the first container.

13. A kit comprising a box with a lid and following components placed therein:
a) a pre-filled syringe of an influenza virus vaccine that elicits a protective immune response to subtypes A and type B of the influenza virus;
b) an isopropyl alcohol pad;
c) a spot adhesive bandage;
d) a pair of gloves; and
e) a CSR (Central Supply Room) wrap.
